(19) United States
(12) Patent Application Publication
(10) Pub. No.: US 2024/0333670 A1
(43) Pub. Date: Oct. 3, 2024

(54) COMMUNICATIONS INTERVENTION SYSTEM AND METHOD

(71) Applicant: Susan Lee Stanford, Fort Walton Beach, FL (US)

(72) Inventor: Susan Lee Stanford, Fort Walton Beach, FL (US)

(21) Appl. No.: 18/454,928

(22) Filed: Aug. 24, 2023

Related U.S. Application Data
(60) Provisional application No. 63/492,347, filed on Mar. 27, 2023.

(51) Int. Cl. H04L 51/063; G06F 40/166; G06F 40/35; G06F 40/56

(52) U.S. Cl. CPC H04L 51/063; G06F 40/166; G06F 40/35; G06F 40/56

(57) ABSTRACT

A method for monitoring an individual client's communications and recommending alternative phrasing includes receiving input from an individual provider serving the client and storing the input in a database of a server; monitoring the client's communications via the client's communications device; intervening in the client's communications when they meet a predetermined set of criteria; storing data regarding the intervention in a database; compiling the data periodically into a report; delivering the report to the client; and automatically communicating an alert including the data to support personnel if the data indicate the client is experiencing an emergency. The input includes settings for monitoring the client's communications and criteria for intervening. administering the settings and criteria are administrated on the communications device. The device communicates with the server by one or more networks. The report includes recommendations for improving the client's communications.

### CROSS-REFERENCE TO RELATED APPLICATION

This application claims the benefit of priority of U.S. provisional application No. 63/492,347, filed Mar. 27, 2023, the contents of which are herein incorporated by reference.

### BACKGROUND OF THE INVENTION

The present invention relates to software and, more particularly, to a communications intervention system and method. This software can be used as an accommodation tool and training system for a disabled person at work, at school, or even when fitting into personal social situations.

Individuals with emotional and/or mental disabilities are often unable to communicate in a socially acceptable and effective manner. These disabled people find this disconnect frustrating, depressing and dangerously destructive to their social, academic, and work lives/careers. Unfortunately, many of these individuals make no sense to other people and fail to properly express themselves. They just cannot get the point across and/or come off aggressive and hostile, accidentally communicating socially, academically, and professionally in unacceptable terms, expressing only anger, frustration, and hostility. It is extremely difficult for the speaker or writer to catch themselves in this pattern until it is too late. They do not realize they have made a mistake until a friend, co-worker, or fellow student is offended.

As can be seen, there is a need for a means of assisting emotionally and mentally disabled individuals to communicate more effectively.

It is imperative to have “in time” intervention during the communication. This way the individual is no longer sabotaging their social life, education and career. The intervention app described herein will help alleviate further depression and destruction and assist mentally and emotionally disabled individuals to succeed in the workforce, etc., keeping them in school and in the workplace instead of on disability or otherwise not contributing to society/the community.

### SUMMARY OF THE INVENTION

One aspect of the present invention includes a method for monitoring an individual client's/patient's communications and recommending alternative phrasing. An application performing the method receives input from an individual provider serving the individual client or patient receiving treatment, wherein the input includes settings for monitoring communications of the client and criteria for intervening, and wherein the input is stored in a database of a server. The application administers the settings and criteria on a client's communications device in communication with the server by one or more networks and monitors the client's communications via the client's communications device, intervening in the client's communications when the client's communications meet a predetermined set of criteria. The application stores data regarding the intervening in the database or in a memory of the client's communications device, compiles the data periodically into a report, including providing recommendations for improving the communications, delivers the report to the client, and automatically communicates an alert including the data to support personnel if the data indicate the client is experiencing an emergency.

These and other features, aspects and advantages of the present invention will become better understood with reference to the following drawings, description, and claims.

### BRIEF DESCRIPTION OF THE DRAWINGS

FIG. 1 is a flow chart of a doctor side of a process according to an embodiment of the present invention;

FIG. 2 is a continuation of FIG. 1;

FIG. 3 is a flowchart of a patient side thereof;

FIG. 4 is a continuation of FIG. 3;

FIG. 5 is a flowchart of a reporting process thereof; and

FIG. 6 is a block diagram of a system according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a software application (app) that monitors an individual's verbal and/or written communication, flags problematic words or phrases, and suggests more suitable alternatives.

The application is sometimes referred to herein as “Decorum.” Decorum generally suggests different words during communication.

The app may be installed on any communications device, including a smart watch, a computer (whether desktop, laptop, or tablet), and a smartphone. The app may produce a glossary of alternative words that the individual may carry with them, such as in book form. The application may be coupled with existing communications apps, such as text or messaging apps, email apps such as Outlook®, voice-to-text applications that listen to speech, etc. The user may scan speeches or letters, using the app to identify red flags in the text and enabling the user to rewrite text or change their tone.

The app may alert the user when their communication is hostile or hyperactive, they talk too much and/or need to stop talking, they need to take a break, they need to call their therapist, or they should change their tone. The app catches problem issues, including anger, all-or-nothing words, and any words that are too passive, vague, or incomprehensible and suggests a phrase or word that guides the user to a nicer way to communicate. For example, the app may advise the individual how a reader may react to a statement phrased in a first manner as compared to how the reader may react to the statement phrased in a second manner.

In some embodiments, the app may be used to identify certain behavioral or communications areas needing more work.

The application can be used in medicine and is designed to comply with the Health Insurance Portability and Accountability Act (HIPAA).

A doctor may review the patient's history, make a diagnosis, and customize the app's settings to identify red flags and make suggestions based on a patient's illness, enabling the patient to learn how to modify their language and actions. The doctor may pick verbal and/or written monitoring. The app may be used to monitor communication for patients with a variety of disorders, such as bipolar disorder, borderline personality disorder, autism, etc.

The app may be configured to enable a doctor to track a client's status as often as moment by moment and/or to monitor the client's health periodically, such as once a month. In some embodiments, the doctor may be notified in the event of an emergency. The app saves interactions and concatenates data to maintain a history so that the client and doctor can see improvement or lack thereof. This app can assist the person's doctor in monitoring progress and the doctor can help adjust settings on the app according to the illness.

Doctors and/or users may add training and games to practice with no risk and to see if there is progress. In some embodiments, the app may be installed in toys.

In some embodiments, the app is envisioned as a potentially mandatory accommodation for employees.

Services may be adapted in real-time based on dynamic learning from aggregated data. Reporting may be performed without compromising the privacy of client information and data or violating applicable laws. The intrinsic codes of the various embodiments provide artificial intelligence and machine learning. The embodiments may particularly implement big data analytics, machine and deep learning, human like reasoning, continuous real-time and deep learning, traceable justification and casual reason for decision outputs, interoperability (e.g., handling logical contradictions, data incompleteness, etc.). The various embodiments are utilized to perform logical and causal reasoning (e.g., inference/reasoning, multi-phase logic, conceptualization, exploration, scenarios/what-ifs, fuzzy logic, and dynamic fight/flight mitigation. The client data may be analyzed utilizing algorithms, pattern analysis, perceptions, recognition/identification, statistical co-occurrence, multi-sensor perceptions, and Bayesian/dissipative recognition.

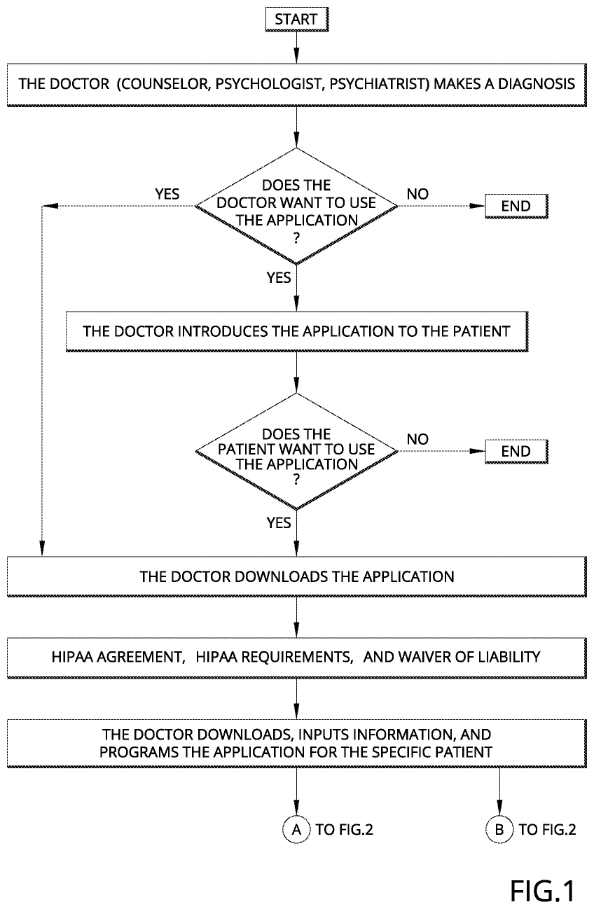

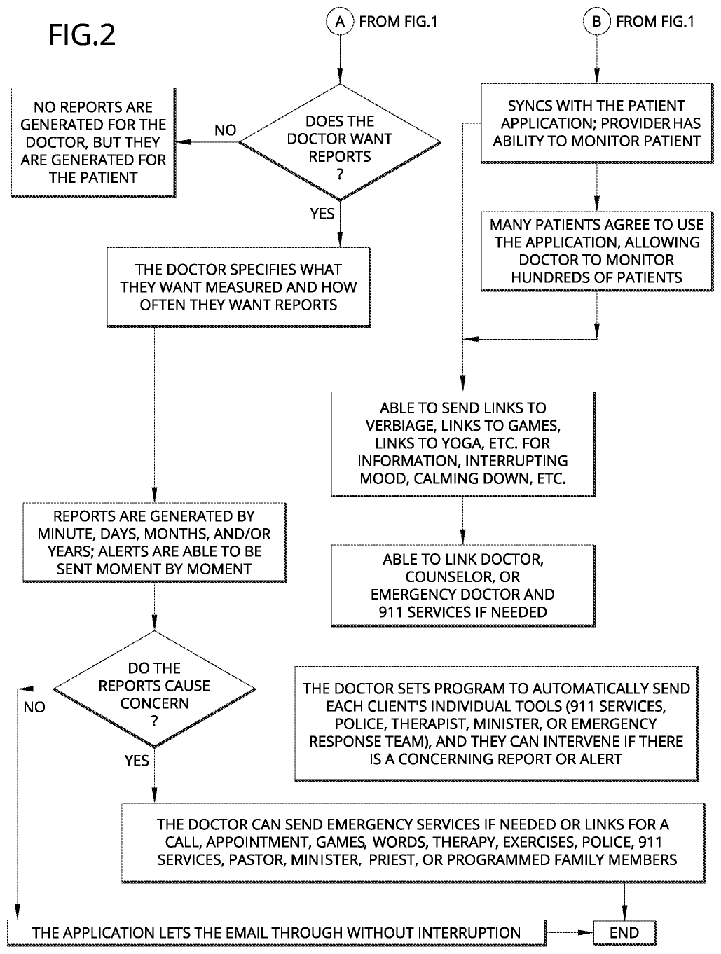

Referring to FIGS. 1 through 7, FIGS. 1 through 5 are flowcharts illustrating various features of a method according to an embodiment of the present invention. FIGS. 1 and 2 show method steps from a doctor's (e.g., a counselor, psychologist, or psychiatrist) perspective using the inventive app. If the doctor chooses to use the app, he or she introduces the app to the patient. If the patient chooses to use the app, the doctor downloads the application, logs in as a provider, and navigates through a HIPAA agreement, meeting HIPAA requirements, as well as a waiver of liability. The patient downloads the app onto their device as well. The doctor customizes the app for the individual patient, entering information and selecting whether to receive reports generated by the app. If the doctor chooses not to receive reports from the app, reports are generated for the patient alone. Otherwise, the doctor selects how often they want to receive reports (e.g., a number of minutes, days, months, and/or years) and what the content of the reports should show. Alerts may also be generated on a moment-to-moment basis. The doctor syncs his or her app with the patient's app, giving the provider access to monitor the patient, send links to vocabulary words, games, yoga and/or other exercise, therapy resources, an appointment scheduler, and support personnel, etc. for information, mood management, and anxiety management. If many patients agree to use the application, a single doctor may monitor potentially hundreds of patients. Support personnel may include, for example, police, 911 services, a pastor, minister, priest or other clergy member, a therapist, and/or programmed family members. The doctor can alert emergency personnel if there is a concerning report or alert. Alternatively, the doctor can set the app to automatically notify 911 services, police, a therapist, a minister, or an emergency response team in an emergency, so that they can intervene.

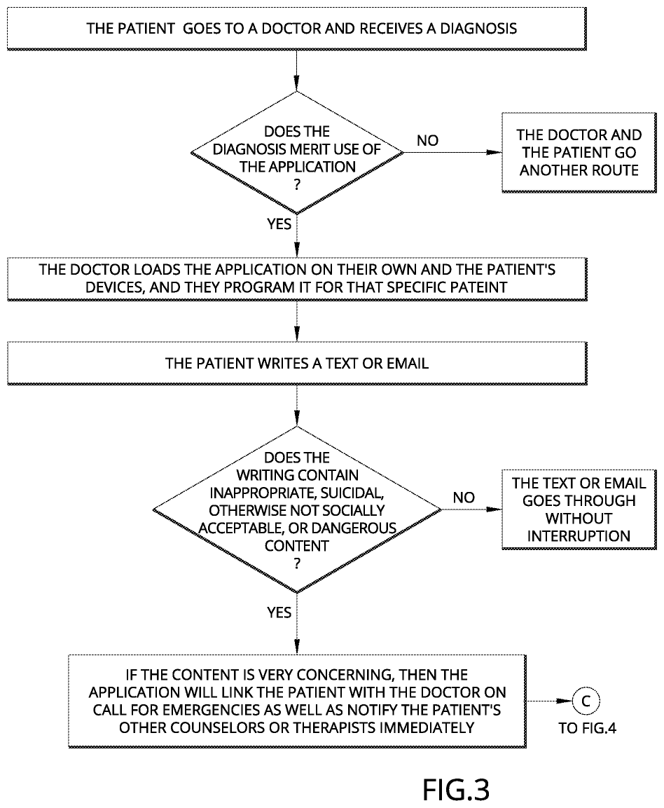

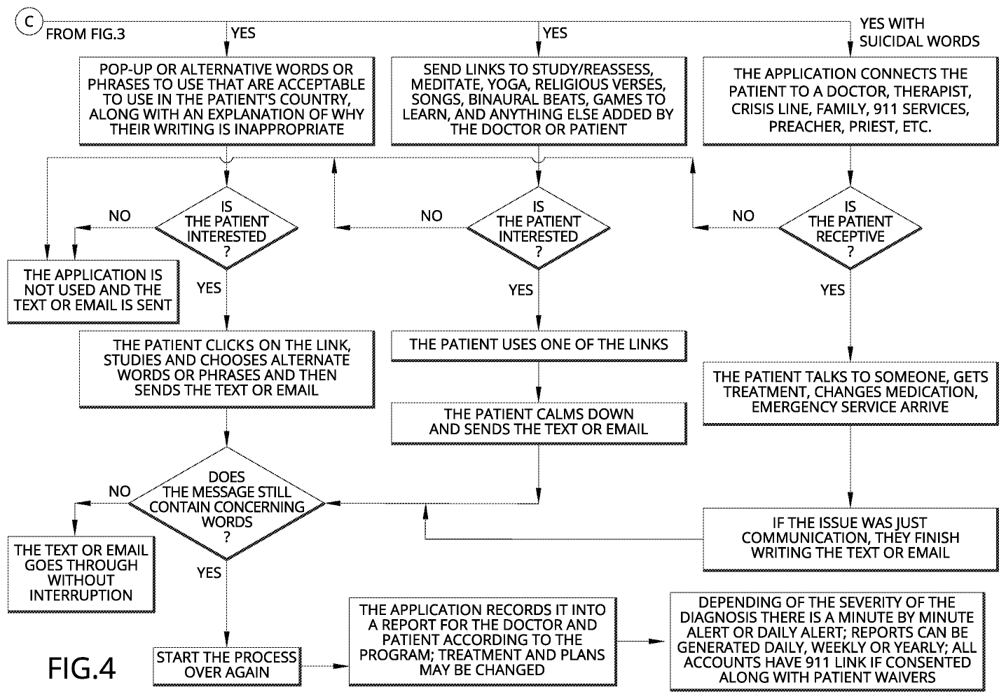

FIGS. 3 and 4 illustrate the method from a patient's perspective. If the patient receives a diagnosis from a doctor which merits use of the app, the patient downloads the app onto their device and the doctor uses their instance of the app to program the patient's app or sets the parameters for the patient's app directly on the patient's device in some cases. When the patient writes a text or email, for example, the app monitors the writing for any inappropriate, suicidal, dangerous, or socially unacceptable content. In the absence of such content, the app does not interrupt the text or email. Otherwise, the app generates a pop-up or inserts alternative words or phrases with an explanation of why the alternative language has been suggested. The app may also provide the patient with links to study or reassess, including content such as religious verses and/or songs, and/or support for meditation, yoga, and games. If the app finds any particularly concerning content, it may link the patient with an on-call emergency doctor and may immediately notify any of the patient's other providers. The patient may choose whether to comply with the app's suggestion or reject it. If the patient rejects the intervention, the communication is sent without further interruption. If the patient accepts the recommendations, they may modify the communication and send it, use the resources to calm down, and/or receive support by talking with someone, changing medication, or having emergency service. The app may reassess the modified communication and repeat the intervention if the content has not improved. The app records the incident for entry into a report and an alert may be generated. The reports may be used to iteratively change the patient's treatment plan.

FIG. 5 illustrates a back-end perspective of the method. The app monitors the patient's communications and generates a report suggesting any weak areas and ways to improve the patient's communications skills and behavioral self-management, which may be delivered to the user and/or the doctor. The app may offer exercises, tasks, and vocabulary to the user, as well as resources on how to self-soothe, such as yoga and ways to determine why the patient may be hostile. particularly concerning reports may lead to inpatient care, additional therapy, changes in medication, and intervention by clergy and/or suicide prevention providers.

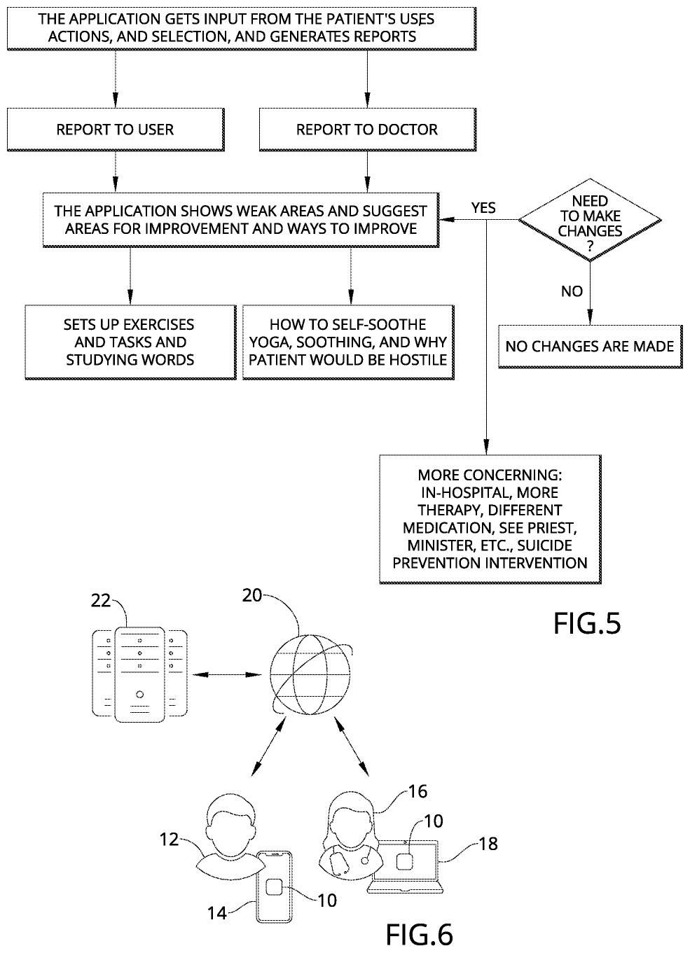

As shown in FIG. 6, a system according to an embodiment of the present invention may include communications devices 14, 18 for the patient 12 and the provider 16, both having the app 10 installed. The devices communicate with a server 22 via the internet or other network 20.

It should be understood, of course, that the foregoing relates to exemplary embodiments of the invention and that modifications may be made without departing from the spirit and scope of the invention as set forth in the following claims.

What is claimed is:

1. A method for monitoring an individual's communications and recommending alternative phrasing, the method comprising:
receiving input from an individual provider serving the individual receiving treatment, wherein the input includes settings for monitoring communications of the individual and criteria for intervening, wherein the input is stored in a database of a server, and administering the settings and criteria on an individual's communications device in communication with the server by one or more networks;
monitoring the individual's communications via the individual's communications device;
intervening in the individual's communications when the individual's communications meet a predetermined set of criteria;
storing data regarding the intervening in the database or in a memory of the individual's communications device;
compiling the data periodically into a report, including providing recommendations for improving the communications;
delivering the report to the individual; and
automatically communicating an alert including the data to support personnel if the data indicate the individual is experiencing an emergency.

2. The method according to claim 1, wherein the intervening comprises automatically recommending the alternative phrasing to the individual.

3. The method according to claim 1, wherein the intervening comprises recommending mood and/or anxiety management techniques to the individual.

4. The method according to claim 1, further comprising delivering the report to the provider.

5. The method according to claim 1, further comprising providing training exercises to the individual to improve communication skills and/or self-management skills.

6. A system for monitoring an individual's communications and recommending alternative phrasing, the system comprising:
a server that communicates with communications devices through a transceiver utilizing one or more networks, the server including a processor that executes a communications monitoring and intervention program stored in a memory to perform the method of claim 1; and
a database in communication with the server that manages the data, the database configured to store accounts of the provider and the individual, the data, and the report.

7. The system according to claim 6, wherein the communications monitoring and intervention program is configured to automatically recommend the alternative phrasing to the individual.

8. The system according to claim 6, wherein the communications devices represent wired or wireless communications and computing devices including the individual's communications device, and wherein the communications devices execute a version of the communications monitoring and intervention program and are utilized by the provider to receive the data.

9. A server comprising:
a processor for executing a set of instructions for monitoring an individual's communications and recommending alternative phrasing; and
a memory for storing the set of instructions for monitoring the individual's communications and recommending alternative phrasing, wherein the set of instructions are executed by the processor to perform the method of claim 1.

10. The server according to claim 9, wherein the set of instructions are further executed to automatically recommend the alternative phrasing to the individual.

11. The server according to claim 10, wherein the server is accessible by the individual's communications device.
